(19) United States
(12) Patent Application Publication
(10) Pub. No.: US 2013/0084313 A1
(43) Pub. Date: Apr. 4, 2013

(54) SUPPLEMENT FOR OSTOMY PATIENTS

(71) Applicant: Stomavite, LLC, North Garden, VA (US)

(72) Inventors: Timothy L. MacDonald, Charlottesville, VA (US); George Sachs, Encino, CA (US)

(73) Assignee: STOMAVITE, LLC, North Garden, VA (US)

(21) Appl. No.: 13/631,827

(22) Filed: Sep. 28, 2012

Related U.S. Application Data
(60) Provisional application No. 61/541,795, filed on Sep. 30, 2011.

(51) Int. Cl. A61K 9/00

(52) U.S. Cl. CPC A61K 9/0002
USPC 424/400; 424/94.61

(57) ABSTRACT

The present application describes a supplement containing an enteric coated alpha-D-galactosidse and the use thereof to treat an ostomy patient.

### FIELD OF THE INVENTION

The present invention relates to supplements for an ostomy patient and a method of treating an ostomy patient with the supplement.

### BACKGROUND OF THE INVENTION

Ileostomy, colostomy, and urostomy are life saving procedures that often lead to impaired quality of life, largely due to irritation at the site of externalization of the functional ileum, colon or neo-bladder. Related issues include uncontrolled passage of gas, leakage of content, and nutrient deficiency, though the latter is more often associated with ileostomy than colostomy.

It is desirable to discover novel supplement to enhance the quality of life for ostomy patients.

### SUMMARY OF THE INVENTION

Described herein is an enteric coated, enzyme-based supplement useful for ostomy patients.

These and other aspects, which will become apparent during the following detailed description, have been achieved by the inventor's discovery of a new supplement for ostomy patients.

### DETAILED DESCRIPTION OF PREFERRED EMBODIMENTS

Alpha-D-galactosidase, which is an active additive in Beano®, is an accepted medication for gas reduction by reducing lactose levels to avoid bacterial fermentation and gas production. However it is administered without gastric acid protection and much is destroyed before entering the small intestine.

We have developed an enzyme-based supplement wherein the enzyme is enterically coated to allow more of the enzyme to survive gastric acids in order that more lactose can be hydrolyzed in the duodenum compared with non-enterically coated enzymes.

Therefore, in an aspect, an ostomy supplement is provided, comprising:
a. a therapeutically effective amount of a first alpha-D-galactosidase; and,
b. a pH 5 enteric coating;

wherein the alpha-D-galactosidase is substantially coated by the enteric coating.

In another aspect, the supplement further comprises:
c. a first pharmaceutically acceptable carrier;

wherein the alpha-D-galactosidase and carrier are substantially coated by the enteric coating.

In another aspect, an ostomy supplement is provided, comprising:
a. a therapeutically effective amount of a first alpha-D-galactosidase; and,
b. a pH 7 enteric coating;

wherein the alpha-D-galactosidase is substantially coated by the enteric coating.

In another aspect, the supplement further comprises:
c. a first pharmaceutically acceptable carrier;

wherein the alpha-D-galactosidase and carrier are substantially coated by the enteric coating.

In another aspect, the supplement further comprises:
d. a therapeutically effective amount of a second alpha-D-galactosidase substantially coated with a pH 5 enteric coating.

In another aspect, the supplement further comprises:
e. a second pharmaceutically acceptable carrier;

wherein the second alpha-D-galactosidase and the second carrier are substantially coated by the pH 5 enteric coating.

The examples provided herein are non-inclusive unless otherwise stated. They include but are not limited to the recited examples.

Examples of the amount of alpha-D-galactosidase present in the supplement include from 500, 600, 700, 800, 900, 1000, 1100, 1200, 1300, 1400, 1500, 1600, 1700, 1800, 1900, to 2000 GaIU.

The enteric coating used in the present invention is selected to provide significant gastroprotection and allow the alpha-D-galactosidase to reach the duodenum as compared to uncoated enzyme. In the duodenum the enzyme is expected to hydrolyse lactose in the diet before being subjected to pancreatic proteases. Examples of the amount of administered alpha-D-galactosidase that reaches the duodenum include 50, 55, 60, 65, 70, 75, 80, 85, 90, 95, 96, 97, 98, 99, to 100%.

It is desirable for the alpha-D-galactosidase, and pharmaceutically acceptable carrier if present, to be substantially coated by the enteric coating. The level of coating desired would be expected to be sufficient to deliver a therapeutically effective amount of alpha-D-galactosidase to the duodenum. Examples of substantially include 50, 55, 60, 65, 70, 75, 80, 85, 90, 95, 96, 97, 98, 99, to 100%.

A pH 5 enteric coating is resistant to hydrolysis at a pH of less than about 5 (i.e., a pH more acidic than 5). A pH 7 enteric coating is resistant to hydrolysis at a pH of less than about 7 (i.e., a pH more acidic than 7). It may be desirable for the present enteric coating to dissolve at a pH of 5, 5.5, 6, 6.5, 7, 7.5, 8, to 8.5. Examples of polymers suitable for enteric coating include:
a. polymethacrylates (e.g., methacrylic acid/ethyl acrylate and methacrylic acid methylmethacrylate co-polymer),
b. cellulose esters (e.g., cellulose acetate phthalate (CAP), cellulose acetate trimellitate (CAT), cellulose acetate succinate (CAS), hydroxypropylmethylcellulose acetate succinate (HPMCAS), hypromellose acetate succinate, and hydroxypropyl methylcellulose phthalate), and
c. polyvinyl derivatives (e.g., polyvinyl acetate phthalate (PVAP)).

A pharmaceutically acceptable carrier is typically an inactive substance that assists in the formulation and/or delivery of an active substance. Carrier includes standard pharmaceutical substances that have been approved by a regulatory agency of the US and/or EU and include those listed in the US and European Pharmacopeias (e.g., talc, silicon dioxide, starch, calcium silicate, mineral oils, vegetable oils, and paraffins).

In another aspect, at least one supplement (e.g., a tablet) is administered once daily to a patient in need thereof. The presence of the enteric coating should provide patients the freedom to ingest the supplement before, during, or after a meal. Patients that may find the supplement useful include those who have had a colostomy or an ileostomy. Additional examples include administering at least 2, 3, 4, or 5 supplements daily. While it may be desirable to administer one or two supplements once per day, a supplement or supplements can also be administed twice or three times per day.

In another aspect, the supplement is divided into two dosages or three dosages.

In another aspect, a package is provided, comprising: at least one supplement. An example of package is a blister pack comprising at least two supplements (e.g., two tablets). The at least two supplements can be package together (e.g., one blister containing two supplements) or separately (e.g., two blisters containing one supplement each). An individual blister pack can comprise 1, 2, 3, 4, 5, 6, 7, 8, 9, 10, 11, 12, or more blisters.

The supplement of the present invention can be formulated into any useful orally administered form, including tablets, capsules, powders, etc.

In another aspect, the supplement, further comprises: a therapeutically effective amount of at least one additional additive, The supplement can further comprise: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 additional additives. Examples of additives include vitamins (e.g., see additives 1-13 below), minerals (e.g., see additives 16-33), natural products (e.g., see additives 14, 15, and 35 below), and un-natural products (e.g., see additive 34 below).

Examples of additives include additives 1-35 below. The ions listed below, when present as an additive in the supplement of the present invention, are in at least one of the various known forms of the additive (e.g., see the examples listed).

| | Additive |
|---|---|
| 1 | Vit A (carotene equiv) |
| 2 | Vit D |
| 3 | Vit E (tocopherol) |
| 4 | Vit K |
| 5 | Vit B1 (thiamin) |
| 6 | Vit B2 (riboflavin) |
| 7 | Vit B3 (niacin) |
| 8 | Vit B5 (pantothenate) |
| 9 | Vit B6 (pyridoxine) |
| 10 | Vit B7 (biotin) |
| 11 | Vit B9 (folic acid) |
| 12 | Vit B12 (cobalamin) |
| 13 | Vit C (ascorbic acid) |
| 14 | Lycopene |
| 15 | Lutein |
| 16 | Ca²⁺ (e.g., Calcium Citrate) |
| 17 | Mg²⁺ (e.g., Magnesium Citrate) |
| 18 | Zn²⁺ (e.g., Zinc Picolinate) |
| 19 | Mn²⁺ (e.g., Manganese amino acid chelate) |
| 20 | Fe²⁺ (e.g., Iron Sulfate) |
| 21 | Se²⁺ (e.g., Selenomethionine) |
| 22 | Cr²⁺ (e.g., Chromium Picolinate) |
| 23 | Mo²⁺ (e.g., Ammonium Molybdate) |
| 24 | K⁺ (e.g., Potassium Gluconate) |
| 25 | Cu⁺ (e.g., Copper Gluconate) |
| 26 | Cl⁻ (e.g., Potassium Chloride) |
| 27 | I⁻ (e.g., Potassium Iodide) |
| 28 | PO₄⁻³ (e.g., Calcium Phosphate) |
| 29 | B³⁺ (e.g., Boron amino acid chelate) |
| 30 | Ni²⁺ (e.g., Nickel Chloride) |
| 31 | Si⁴⁺ (e.g., Orthosilicic Acid) |
| 32 | Sn²⁺ (e.g., Tin Chloride) |
| 33 | V^{x+} (e.g.,Vanadyl Sulfate) |
| 34 | Simethicone |
| 35 | Aloe Vera |

Examples of therapeutically effective amounts of additional additives include:

| | Additive | Amount |
|---|---|---|
| 1 | Vit A (carotene equiv) | 1000, 1500, 2000, 2500, 3000, to 3500 IU |
| 2 | Vit D | 100, 150, 200, 250, 300, 350, 400, 450, to |
| | | 500 IU |
| 3 | Vit E (tocopherol) | 50, 55, 60, 65, 70, 75, 80, 85, 90, 95, to 100 |
| | | IU |
| 4 | Vit K | 15, 20, 25, 30, to 35 mcg |
| 5 | Vit B1 (thiamin) | 0.1, 0.2, 0.3, 0.4, 0.5, 0.6, 0.7, 0.8, 0.9 1.0, |
| | | 1.1, 1.2, 1.3, 1.4, 1.5, 1.6, 1.7, 1.8, |
| | | 1.9, to 2.0 mg |
| 6 | Vit B2 (riboflavin) | 0.1, 0.2, 0.3, 0.4, 0.5, 0.6, 0.7, 0.8, 0.9 1.0, |
| | | 1.1, 1.2, 1.3, 1.4, 1.5, 1.6, 1.7, |
| | | 1.8, 1.9, to 2.0 mg |
| 7 | Vit B3 (niacin) | 5, 10, 15, 20, to 25 mg |
| 8 | Vit B5 (pantothenate) | 5, 10, 15, 20, to 25 mg |
| 9 | Vit B6 (pyridoxine) | 1, 2, 3, 4, to 5 mg |
| 10 | Vit B7 (biotin) | 15, 20, 25, 30, 35, 40, to 45 mcg |
| 11 | Vit B9 (folic acid) | 50, 100, 150, 200, 250, 300, to 350 mcg |
| 12 | Vit B12 (cobalamin) | 1, 2, 3, 4, 5, 6, 7, to 8 mcg |
| 13 | Vit C (ascorbic acid) | 50, 100, 150, 200, to 250 mg |
| 14 | Lycopene | 100, 200, 300, 400, 500 mcg |
| 15 | Lutein | 150, 200, 250, 300, to 350 mcg |
| 16 | Ca²⁺ | 150, 200, 250, 300, to 350 mg |
| 17 | Mg²⁺ | 50, 100, 150, 200, 250, 300, to 350 mg |
| 18 | Zn²⁺ | 60, 70, 80, 90, to 100 mg |
| 19 | Mn²⁺ | 0.1, 0.2, 0.3., 0.4, 0.5, 0.6, 0.7, 0.8, 0.9, to 1 |
| | | mg |
| 20 | Fe²⁺ | 0.1, 0.2, 0.3., 0.4, 0.5, 0.6, 0.7, 0.8, 0.9, to 1 |
| | | mg |
| 21 | Se²⁺ | 25, 50, 75, 100, 125, 150, 175, to 200 mcg |
| 22 | Cr²⁺ | 40, 60, 80, 100, 120, 140, 160, 180, to 200 |
| | | mcg |
| 23 | Mo²⁺ | 10, 20, 30, 40, 50, 60, 70, to 80 mcg |
| 24 | K⁺ | 100, 200, 300, 400, 500, 600, to 700 mg |
| 25 | Cu⁺ | 50, 100, 150, 200, 250, 300, to 350 mcg |
| 26 | Cl⁻ | 60, 62, 64, 66, 68, 70, 72, 74, 76, 78, 80, 82, |
| | | to 84 mg |
| 27 | I⁻ | 60, 65, 70, 75, 80, 85, to 90 mcg |
| 28 | Phosphate | 90, 100, 110, 120, to 130 mg |
| 29 | Bo³⁺ | 60, 65, 70, 75, 80, 85, to 90 mcg |
| 30 | Ni²⁺ | 2, 3, 4, 5, 6, 7, to 8 mcg |
| 31 | Si⁴⁺ | 0.5, 1, 1.5, 2, 2.5, 3, to 3.5 mg |
| 32 | Sn²⁺ | 2, 3, 4, 5, 6, 7, to 8 mcg |
| 33 | V | 2, 3, 4, 5, 6, 7, to 8 mcg |
| 34 | Simethicone | 50, 100, 150, 200, 250, 300, to 350 mg |
| 35 | Aloe | 100, 200, 300, 400, 500, 600, 700, 800, 900, |
| | | to 1000 mg |

The amounts shown below are amounts for daily intake. If it is deemed desirable to divide the supplement into two dosages, then the amount present in each supplement would be half the daily intake. This follows if three, four, or more daily dosages are desired.

Other features of the invention will become apparent in the course of the following descriptions of exemplary embodiments that are given for illustration of the invention and are not intended to be limiting thereof.

### EXAMPLES

### Example 1

A tablet containing:

| Material | Amount | RDA (%) |
|---|---|---|
| Alpha-D-galactosidase¹ | 1000 GaIU | * |

*No RDA suggested.
¹pH 5.0 enteric coated.

A tablet containing the above ingredients would be dosed once per day. If the size of the table is undesirable, then two or three tables daily can be used. If two tablets are desired, then the amounts provided above would be divided in half for each tablet. If three tables are desired, then the amounts provided about would be divided by three for each tablet.

### Example 2

A tablet (or tablets) containing the following additives.

| Material | Amount | | RDA (%) |
|---|---|---|---|
| Vit A (carotene equiv) | 2500 | IU | 50 |
| Vit D | 300 | IU | 75 |
| Vit E (tocopherol) | 75 | IU | 250 |
| Vit K | 25 | mcg | 31 |
| Vit B1 (thiamin) | 1.0 | mg | 67 |
| Vit B2 (riboflavin) | 1.0 | mg | 67 |
| Vit B3 (niacin) | 15 | mg | 75 |
| Vit B5 (pantothenate) | 5.0 | mg | 50 |
| Vit B6 (pyridoxine) | 3.0 | mg | 75 |
| Vit B7 (biotin) | 30 | mcg | 10 |
| Vit B9 (folic acid) | 200 | mcg | 50 |
| Vit B12 (cobalamin) | 4 | mcg | 67 |
| Vit C (ascorbic acid) | 150 | mg | 250 |
| Lycopene | 300 | mcg | * |
| Lutein | 250 | mcg | * |
| Ca²⁺ | 250 | mg | 25 |
| Mg²⁺ | 200 | mg | 50 |
| Zn²⁺ | 80 | mg | 50 |
| Mn²⁺ | 0.5 | mg | 50 |
| Fe²⁺ | 0.5 | mg | 6 |
| Se²⁺ | 100 | mcg | 100 |
| Cr²⁺ | 120 | mcg | 100 |
| Mo²⁺ | 40 | mcg | 50 |
| K⁺ | 400 | mg | 10 |
| Cu⁺ | 200 | mcg | 10 |
| Cl⁻ | 72 | mg | 2 |
| I⁻ | 75 | mcg | 50 |
| PO₄³⁺ | 110 | mg | 11 |
| B³⁺ | 75 | mcg | * |
| Ni²⁺ | 5.0 | mcg | * |
| Si⁴⁺ | 2 | mg | * |
| Sn²⁺ | 5.0 | mcg | * |
| V^{x+} (undisclosed charge) | 5.0 | mcg | * |
| Simethicone | 200 | mg | * |
| Alpha-D-galactosidase¹ | 1000 | GaIU | * |

*No RDA suggested.
¹pH 5.0 enteric coated.

A tablet containing the above ingredients would be dosed once per day. If the size of the table is undesirable, then two or three tables daily can be used. If two tablets are desired, then the amounts provided above would be divided in half for each tablet. If three tables are desired, then the amounts provided about would be divided by three for each tablet.

### Example 3

The tablet (or tablets) of Example 2 containing the following additional additive.

| | | |
|---|---|---|
| Aloe Vera | 500 mg | * |

*No RDA suggested.

### Example 4

A tablet containing:

| Material | Amount | RDA (%) |
|---|---|---|
| Alpha-D-galactosidase¹ | 1000 GaIU | * |

*No RDA suggested.
¹pH 7.0 enteric coated.

A tablet containing the above ingredients would be dosed once per day. If the size of the table is undesirable, then two or three tables daily can be used. If two tablets are desired, then the amounts provided above would be divided in half for each tablet. If three tables are desired, then the amounts provided about would be divided by three for each tablet.

### Example 5

A tablet (or tablets) containing the following additives.

| Material | Amount | | RDA (%) |
|---|---|---|---|
| Vit A (carotene equiv) | 2500 | IU | 50 |
| Vit D | 300 | IU | 75 |
| Vit E (tocopherol) | 75 | IU | 250 |
| Vit K | 25 | mcg | 31 |
| Vit B1 (thiamin) | 1.0 | mg | 67 |
| Vit B2 (riboflavin) | 1.0 | mg | 67 |
| Vit B3 (niacin) | 15 | mg | 75 |
| Vit B5 (pantothenate) | 5.0 | mg | 50 |
| Vit B6 (pyridoxine) | 3.0 | mg | 75 |
| Vit B7 (biotin) | 30 | mcg | 10 |
| Vit B9 (folic acid) | 200 | mcg | 50 |
| Vit B12 (cobalamin) | 4 | mcg | 67 |
| Vit C (ascorbic acid) | 150 | mg | 250 |
| Lycopene | 300 | mcg | * |
| Lutein | 250 | mcg | * |
| Ca²⁺ | 250 | mg | 25 |
| Mg²⁺ | 200 | mg | 50 |
| Zn²⁺ | 80 | mg | 50 |
| Mn²⁺ | 0.5 | mg | 50 |
| Fe²⁺ | 0.5 | mg | 6 |
| Se²⁺ | 100 | mcg | 100 |
| Cr²⁺ | 120 | mcg | 100 |
| Mo²⁺ | 40 | mcg | 50 |
| K⁺ | 400 | mg | 10 |
| Cu⁺ | 200 | mcg | 10 |
| Cl⁻ | 72 | mg | 2 |
| I⁻ | 75 | mcg | 50 |
| PO₄³⁺ | 110 | mg | 11 |
| B³⁺ | 75 | mcg | * |
| Ni²⁺ | 5.0 | mcg | * |
| Si⁴⁺ | 2 | mg | * |
| Sn²⁺ | 5.0 | mcg | * |
| V^{x+} (undisclosed charge) | 5.0 | mcg | * |
| Simethicone | 200 | mg | * |
| Alpha-D-galactosidase¹ | 1000 | GaIU | * |

*No RDA suggested.
¹pH 7.0 enteric coated.

A tablet containing the above ingredients would be dosed once per day. If the size of the table is undesirable, then two or three tables daily can be used. If two tablets are desired, then the amounts provided above would be divided in half for each tablet. If three tables are desired, then the amounts provided about would be divided by three for each tablet.

### Example 6

The tablet (or tablets) of Example 5 containing the following additional additive.

| | | |
|---|---|---|
| Aloe Vera | 500 mg | * |

*No RDA suggested.

### Example 7

A tablet containing:

| Material | Amount | RDA (%) |
|---|---|---|
| Alpha-D-galactosidase¹ | 500 GaIU | * |
| Alpha-D-galactosidase² | 500 GaIU | * |

*No RDA suggested.
¹pH 5.0 enteric coated.
²pH 7.0 enteric coated.

A tablet containing the above ingredients would be dosed once per day. If the size of the table is undesirable, then two or three tables daily can be used. If two tablets are desired, then the amounts provided above would be divided in half for each tablet. If three tables are desired, then the amounts provided about would be divided by three for each tablet.

### Example 8

A tablet (or tablets) containing the following additives.

| Material | Amount | | RDA (%) |
|---|---|---|---|
| Vit A (carotene equiv) | 2500 | IU | 50 |
| Vit D | 300 | IU | 75 |
| Vit E (tocopherol) | 75 | IU | 250 |
| Vit K | 25 | mcg | 31 |
| Vit B1 (thiamin) | 1.0 | mg | 67 |
| Vit B2 (riboflavin) | 1.0 | mg | 67 |
| Vit B3 (niacin) | 15 | mg | 75 |
| Vit B5 (pantothenate) | 5.0 | mg | 50 |
| Vit B6 (pyridoxine) | 3.0 | mg | 75 |
| Vit B7 (biotin) | 30 | mcg | 10 |
| Vit B9 (folic acid) | 200 | mcg | 50 |
| Vit B12 (cobalamin) | 4 | mcg | 67 |
| Vit C (ascorbic acid) | 150 | mg | 250 |
| Lycopene | 300 | mcg | * |
| Lutein | 250 | mcg | * |
| Ca²⁺ | 250 | mg | 25 |
| Mg²⁺ | 200 | mg | 50 |
| Zn²⁺ | 80 | mg | 50 |
| Mn²⁺ | 0.5 | mg | 50 |
| Fe²⁺ | 0.5 | mg | 6 |
| Se²⁺ | 100 | mcg | 100 |
| Cr²⁺ | 120 | mcg | 100 |
| Mo²⁺ | 40 | mcg | 50 |
| K⁺ | 400 | mg | 10 |
| Cu⁺ | 200 | mcg | 10 |
| Cl⁻ | 72 | mg | 2 |
| I⁻ | 75 | mcg | 50 |
| PO₄³⁺ | 110 | mg | 11 |
| B³⁺ | 75 | mcg | * |
| Ni²⁺ | 5.0 | mcg | * |
| Si⁴⁺ | 2 | mg | * |
| Sn²⁺ | 5.0 | mcg | * |
| V^{x+} (undisclosed charge) | 5.0 | mcg | * |
| Simethicone | 200 | mg | * |
| Alpha-D-galactosidase¹ | 500 | GaIU | * |
| Alpha-D-galactosidase² | 500 | GaIU | * |

*No RDA suggested.
¹pH 5.0 enteric coated.
²pH 7.0 enteric coated.

A tablet containing the above ingredients would be dosed once per day. If the size of the table is undesirable, then two or three tables daily can be used. If two tablets are desired, then the amounts provided above would be divided in half for each tablet. If three tables are desired, then the amounts provided about would be divided by three for each tablet.

### Example 9

The tablet (or tablets) of Example 8 containing the following additional additive.

| | | |
|---|---|---|
| Aloe Vera | 500 mg | * |

*No RDA suggested

Numerous modifications and variations of the invention are possible in light of the above teachings. It is therefore to be understood that within the scope of the appended claims, the invention may be practiced otherwise that as specifically described herein.

What is claimed is:

1. An ostomy supplement, comprising:
a. a therapeutically effective amount of alpha-D-galactosidase; and,
b. a pH 5 enteric coating;
wherein the alpha-D-galactosidase is substantially coated by the enteric coating.

2. The supplement of claim 1, wherein 1000 GaIU of alpha-D-galactosidase is present.

3. The supplement of claim 2, further comprising: a pharmaceutically acceptable carrier, wherein the alpha-D-galactosidase and carrier are substantially coated by the enteric coating.

4. The supplement of claim 1, further comprising: a therapeutic amount of at least one additive selected from vitamins, minerals, natural products, and un-natural products.

5. The supplement of claim 1, further comprising: therapeutic amount of at least one additive selected from additives 1-35 below and in the amount shown:

| | Additive | Amount | |
|---|---|---|---|
| 1 | Vit A (carotene equiv) | 1000-3500 | IU |
| 2 | Vit D | 100-500 | IU |
| 3 | Vit E (tocopherol) | 50-100 | IU |
| 4 | Vit K | 15-35 | mcg |
| 5 | Vit B1 (thiamin) | 0.1-2.0 | mg |
| 6 | Vit B2 (riboflavin) | 0.1-2.0 | mg |
| 7 | Vit B3 (niacin) | 5-25 | mg |
| 8 | Vit B5 (pantothenate) | 5-25 | mg |
| 9 | Vit B6 (pyridoxine) | 1-5 | mg |
| 10 | Vit B7 (biotin) | 15-45 | mcg |
| 11 | Vit B9 (folic acid) | 50-350 | mcg |
| 12 | Vit B12 (cobalamin) | 1-8 | mcg |
| 13 | Vit C (ascorbic acid) | 50-250 | mg |
| 14 | Lycopene | 100-500 | mcg |
| 15 | Lutein | 150-350 | mcg |
| 16 | Ca²⁺ | 150-350 | mg |
| 17 | Mg²⁺ | 50-350 | mg |
| 18 | Zn²⁺ | 60-100 | mg |
| 19 | Mn²⁺ | 0.1-1 | mg |
| 20 | Fe²⁺ | 0.1-1 | mg |
| 21 | Se²⁺ | 25-200 | mcg |
| 22 | Cr²⁺ | 40-200 | mcg |
| 23 | Mo²⁺ | 10-80 | mcg |
| 24 | K⁺ | 100-700 | mg |
| 25 | Cu⁺ | 50-350 | mcg |
| 26 | Cl⁻ | 60-84 | mg |
| 27 | I⁻ | 60-90 | mcg |
| 28 | Phosphate | 90-130 | mg |
| 29 | Bo³⁺ | 60-90 | mcg |
| 30 | Ni²⁺ | 2-8 | mcg |
| 31 | Si⁴⁺ | 0.5-3.5 | mg |
| 32 | Sn²⁺ | 2-8 | mcg |
| 33 | V | 2-8 | mcg |
| 34 | Simethicone | 50-350 | mg |
| 35 | Aloe vera | 100-1000 | mg |

6. The supplement of claim 5, further comprising: a pharmaceutically acceptable carrier, wherein the alpha-D-galactosidase and carrier are substantially coated by the enteric coating.

7. A method of treating an ostomy patient, comprising: administering a therapeutically effective amount of a supplement to a patient in need thereof, the supplement, comprising:
a. a therapeutically effective amount of alpha-D-galactosidase; and,
b. a pH 5 enteric coating;
wherein the alpha-D-galactosidase is substantially coated by the enteric coating.

8. An ostomy supplement, comprising:
a. a therapeutically effective amount of a first alpha-D-galactosidase; and,
b. a pH 7 enteric coating;
wherein the alpha-D-galactosidase is substantially coated by the enteric coating.

9. The supplement of claim 10, wherein 1000 GaIU of alpha-D-galactosidase is present.

10. The supplement of claim 9, further comprising: a first pharmaceutically acceptable carrier, wherein the first alpha-D-galactosidase and carrier are substantially coated by the enteric coating.

11. The supplement of claim 10, further comprising: a therapeutically effective amount of a second alpha-D-galactosidase substantially coated with a pH 5 enteric coating

12. The supplement of claim 11, further comprising: a second pharmaceutically acceptable carrier, wherein the second alpha-D-galactosidase and second carrier are substantially coated by the pH 5 enteric coating.

13. The supplement of claim 8, further comprising: a therapeutic amount of at least one additive selected from vitamins, minerals, natural products, and un-natural products.

14. The supplement of claim 8, further comprising: therapeutic amount of at least one additive selected from additives 1-35 below and in the amount shown:

| | Additive | Amount | |
|---|---|---|---|
| 36 | Vit A (carotene equiv) | 1000-3500 | IU |
| 37 | Vit D | 100-500 | IU |
| 38 | Vit E (tocopherol) | 50-100 | IU |
| 39 | Vit K | 15-35 | mcg |
| 40 | Vit B1 (thiamin) | 0.1-2.0 | mg |
| 41 | Vit B2 (riboflavin) | 0.1-2.0 | mg |
| 42 | Vit B3 (niacin) | 5-25 | mg |
| 43 | Vit B5 (pantothenate) | 5-25 | mg |
| 44 | Vit B6 (pyridoxine) | 1-5 | mg |
| 45 | Vit B7 (biotin) | 15-45 | mcg |
| 46 | Vit B9 (folic acid) | 50-350 | mcg |
| 47 | Vit B12 (cobalamin) | 1-8 | mcg |
| 48 | Vit C (ascorbic acid) | 50-250 | mg |
| 49 | Lycopene | 100-500 | mcg |
| 50 | Lutein | 150-350 | mcg |
| 51 | Ca²⁺ | 150-350 | mg |
| 52 | Mg²⁺ | 50-350 | mg |
| 53 | Zn²⁺ | 60-100 | mg |
| 54 | Mn²⁺ | 0.1-1 | mg |
| 55 | Fe²⁺ | 0.1-1 | mg |
| 56 | Se²⁺ | 25-200 | mcg |
| 57 | Cr²⁺ | 40-200 | mcg |
| 58 | Mo²⁺ | 10-80 | mcg |
| 59 | K⁺ | 100-700 | mg |
| 60 | Cu⁺ | 50-350 | mcg |
| 61 | Cl⁻ | 60-84 | mg |
| 62 | I⁻ | 60-90 | mcg |
| 63 | Phosphate | 90-130 | mg |
| 64 | Bo³⁺ | 60-90 | mcg |
| 65 | Ni²⁺ | 2-8 | mcg |
| 66 | Si⁴⁺ | 0.5-3.5 | mg |
| 67 | Sn²⁺ | 2-8 | mcg |
| 68 | V | 2-8 | mcg |
| 69 | Simethicone | 50-350 | mg |
| 70 | Aloe vera | 100-1000 | mg |

15. The supplement of claim 19, further comprising: a first pharmaceutically acceptable carrier, wherein the alpha-D-galactosidase and first carrier are substantially coated by the enteric coating.

16. A method of treating an ostomy patient, comprising: administering a therapeutically effective amount of a supplement to a patient in need thereof, the supplement, comprising:
a. a therapeutically effective amount of a first alpha-D-galactosidase; and,
b. a pH 7 enteric coating;
wherein the alpha-D-galactosidase is substantially coated by the enteric coating.

17. The method of claim 16, wherein the supplement, further comprises: a first pharmaceutically acceptable carrier, wherein the first alpha-D-galactosidase and first carrier are substantially coated by the enteric coating.

18. The method of claim 16, wherein the supplement further comprises: a therapeutically effective amount of a second alpha-D-galactosidase substantially coated with a pH 5 enteric coating

19. The method of claim 16, wherein the supplement further comprises: a therapeutically effective amount of a second alpha-D-galactosidase and a second pharmaceutically acceptable carrier, wherein the second alpha-D-galactosidase and second carrier are substantially coated by the pH 5 enteric coating.
